(19) United States
(12) Patent Application Publication
(10) Pub. No.: US 2010/0284558 A1
(43) Pub. Date: Nov. 11, 2010

(54) Earmuff and Headphone

(76) Inventors: Tominori Kimura, Tokyo (JP); Kouhei Yoshida, Tokyo (JP)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/774,236

(22) Filed: May 5, 2010

(30) Foreign Application Priority Data
May 8, 2009 (JP) 2009-113333

(51) Int. Cl. H04R 9/06; A61F 11/14

(52) U.S. Cl. 381/333; 128/866

(57) ABSTRACT

An earmuff includes: a casing; a baffle board; and an ear pad including an engagement member, the engagement member being an outwardly extended part of a skin member of the ear pad. The ear pad is fixed at a front of the baffle board with the engagement member constrained to an outer surface of either of the baffle board and the casing or both by a ring-shaped elastic member, and the ear pad fixed and the baffle board define a front air chamber.

### BACKGROUND OF THE INVENTION

1. Field of the Invention

The present invention relates to an earmuff and a headphone, and more specifically to an earmuff and a headphone having a front air chamber of a larger volume to achieve higher sound insulation against external noise.

2. Description of the Related Art

A conventional earmuff is exemplary shown in a cross-sectional view of FIG. 10. As shown in FIG. 10, in an earmuff 60, an ear pad 63 is fixed to a baffle board 62 that covers an opening of an ear cup 61. A front air chamber 64 is a space surrounded by the baffle board 62 and the ear pad 63.

The baffle board 62 has a shape in which two torus-shaped members having different outer diameters are separated in an axial direction. The front torus-shaped member has a flange 621 formed on an outer periphery. A flap 631 that is a part of a skin member of the ear pad 63 covers the flange 621.

The ear pad 63 is fixed to the baffle board 62 by the flap 631 covering the flange 621. A groove 622 is formed on the entire circumference of the baffle board 62 due to the flange 621.

In the conventional earmuff 60, a gap is likely to be formed at a portion at which the ear pad 63 and the baffle board 62 are in contact with each other. External noise enters the front air chamber 64 through the gap to degrade sound insulation. A headphone is known that solves the problem to achieve improved sound insulation against external noise (see, for example, Japanese Patent Application Publication No. 2009-17176).

A front air chamber plays a significant roll in improving sound insulation of an earmuff. A larger volume of a front air chamber provides higher sound insulation. In the conventional earmuff 60 as shown in FIG. 10, the volume of the front air chamber 64 is compromised as much as the depth of the groove 622. Thus, the volume of the front air chamber 64 and the sound insulation is limited. The headphone disclosed in Japanese Patent Application Publication No. 2009-17176 uses a fixed member of an ear pad to prevent the volume of the front air chamber from being compromised according to the shape of the baffle board as in the conventional example. Unfortunately, such a headphone has a complex structure with increased number of parts.

### SUMMARY OF THE INVENTION

In view of the above, an object of the present invention is to provide an earmuff having higher sound insulation by achieving a larger volume of a front air chamber with: a flap of an ear pad covering an outer peripheral surface of a baffle board; and an elastic ring-shaped member being provided on the outer surface of the flap to fixedly adhere the flap to the outer peripheral surface of the baffle board.

An aspect of the present invention is an earmuff including: a casing; a baffle board; and an ear pad including an engagement member, the engagement member being an outwardly extended part of a skin member of the ear pad. The ear pad is fixed at a front of the baffle board with the engagement member constrained to an outer surface of either of the baffle board and the casing or both by a ring-shaped elastic member, and the ear pad fixed and the baffle board define a front air chamber.

In another embodiment of the earmuff, a supporting member is provided at an end of the outer surface. The supporting member is in contact with a rear surface of the ear pad to prevent the ear pad form sinking into the front air chamber.

In still another embodiment of the earmuff, a supporting member having air permeability is provided on a front surface of the baffle board, and the rear surface of the ear pad is in contact with the supporting member so that the ear pad does not sink into the front air chamber.

In yet still another embodiment of the earmuff, a groove in which the engagement member is constrained is formed on an end of the outer surface.

In yet still another embodiment of the earmuff, the ring-shaped elastic member and the engagement member are integrated.

Another aspect of the present invention is a headphone including a speaker unit provided on a baffle board, the speaker unit outputting an audible signal. An ear pad and the baffle board are any of the above described ear pads and the baffle boards.

In the present invention, the ear pad can be fixedly adhered to the baffle board without compromising the volume of the front air chamber. Thus, an earmuff and a headphone with improved sound insulation can be obtained. Alternatively, if the volume of the front air chamber is as same the conventional counterpart, an earmuff and a headphone formed of a thinner casing so as to be lighter in weight can be obtained.

### BRIEF DESCRIPTION OF THE DRAWINGS

FIG. 1 is a longitudinal sectional view of an embodiment of an earmuff according to the present invention;

FIG. 2 is a longitudinal sectional enlarged view of a part of the earmuff in FIG. 1;

FIG. 3 is a graph depicting a relationship between a volume of a front air chamber and sound insulation effect of the earmuff in FIG. 1;

FIG. 4 is a longitudinal sectional enlarged view of a portion of another embodiment of the earmuff according to the present invention;

FIG. 5 is a longitudinal sectional enlarged view of a portion of still another embodiment of the earmuff according to the present invention;

FIG. 6 is a longitudinal cross-sectional view of another embodiment of an earmuff according to the present invention;

FIG. 7 is a longitudinal sectional enlarged view of a portion of the earmuff illustrated in FIG. 6;

FIG. 8 is a longitudinal sectional enlarged view of a portion of another embodiment of the earmuff according to the present invention;

FIG. 9 is a longitudinal sectional enlarged view of a portion of still another embodiment of the earmuff according to the present invention; and

FIG. 10 is a longitudinal cross-sectional view of a conventional earmuff.

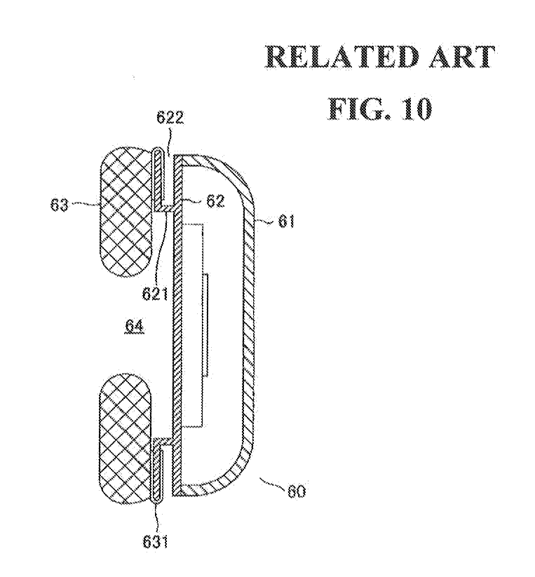

### DETAILED DESCRIPTION OF THE PREFERRED EMBODIMENTS

### First Embodiment

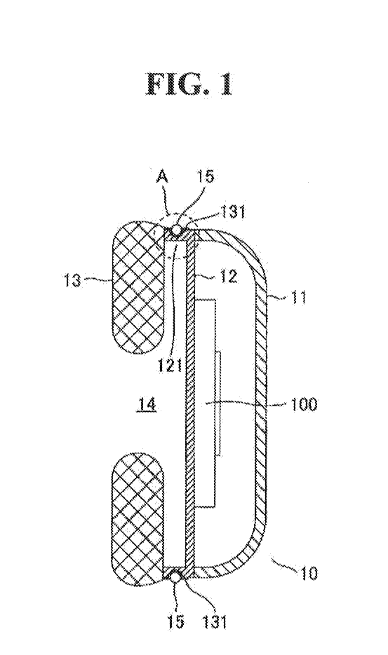

An embodiment of an earmuff according to the present invention is described below with reference to some of the drawings. FIG. 1 is a longitudinal sectional view exemplary illustrating an earmuff according to the present invention. In an earmuff 10 illustrated in FIG. 1, a baffle board 12 is fixed at an opening of an ear cup 11, and an ear pad 13 is fixed to the baffle board 12. The space surrounded by the baffle board 12 and the ear pad 13 is a front air chamber 14.

The baffle board 12 is a substantially circular member covering the opening of the ear cup 11. A rib 121 having a certain height is integrally formed on a peripheral surface of the baffle board 12. A flap 131 is a part of a skin member of the ear pad 13 serving as an engaging unit and is engaged to the periphery of the rib 121 by being constricted with an elastic force of a ring-shaped engagement member 15.

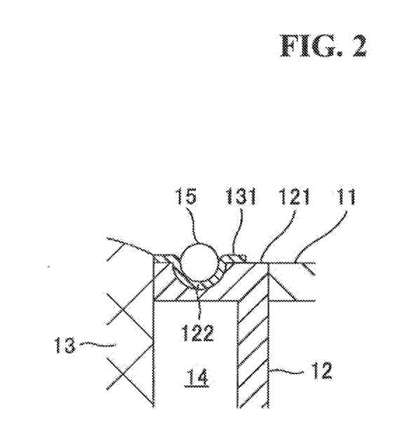

FIG. 2 is an enlarged view of a portion defined by the dotted circle in FIG. 1. As illustrated in FIG. 2, the rib 121 has, on the entire circumference, a groove 122 deep enough to receive the engagement member 15. The flap 131 which is an extended portion of the skin member of the ear pad 13, covers the rib 121. The engagement member 15 is provided on the flap 131 and is fit in the groove 122 so that the flap 131 is pushed against the groove 122 by the elastic force of the engagement member 15. Thus, the ear pad 13 is fixedly adhered to the rib 121 and the ear pad 13 is fixed to the baffle board 12.

As described above, the ear pad 13 can be fixed to the baffle board 12 with the engagement member 15 and the rib 121. Thus, an earmuff having excellent sound insulation can be obtained with the front air chamber 14 of a larger volume that is not compromised by the shape of the baffle board 12. If the volume of the front air chamber is only required to be as same as the conventional counterpart, the baffle board 12 can be made thinner with a shorter rib 121. Thus, with the present invention, an earmuff thinner as a whole can be obtained that provides sound insulating property of a same level as that of the conventional earmuff.

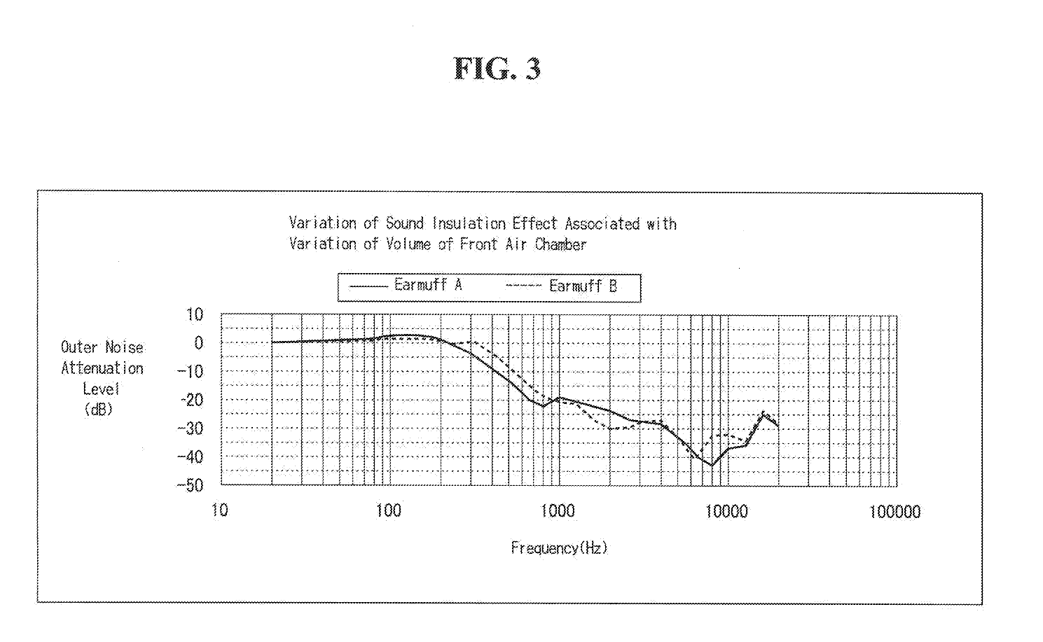

Sound insulating property of the earmuff according to the present invention is described with reference to FIG. 3. FIG. 3 depicts sound insulating properties of two different earmuffs A and B having volumes of about 25 cc and 15 cc, respectively. In FIG. 3: the horizontal axis which is a logarithmic axis, represents frequency; while the vertical axis represents an outer noise attenuation level.

As illustrated in FIG. 3, the earmuffs A and B can both attenuate external noise with high frequencies. Nevertheless, compared with the earmuff B, the earmuff A can more effectively attenuate noise with lower frequencies, and thus exerts higher sound insulating property.

In FIG. 3, frequencies of an external noise that are attenuated by the earmuff A and B for the level of −3 dB are about 250 Hz and 350 Hz, respectively. Therefore, the earmuff A of a volume of the front air chamber larger than that of the earmuff B can exert higher sound insulating property against a noise at lower frequencies, thereby exerting higher sound insulating property against the main frequency band of external noise.

As described above, a larger front air chamber can be obtained by modifying the shape of the baffle board and effectively fixing the ear pad to the baffle board with the engagement member as in the earmuff of the present invention. Thus, an earmuff exerting higher sound insulating property than the conventional counterpart can be obtained. If the sound insulating property is as same as the conventional counterpart, the rib 121 of the baffle board 12 can be shorter, i.e., the baffle board can be thinner, to obtain an earmuff thinner and lighter as a whole.

In other words, the earmuff according to the present invention has the ear pad 13 constrained to be fixed on the outer periphery of the baffle board 12. Therefore, the ear pad can be fixedly adhered to the baffle board without compromising the volume of the front air chamber 14 for improving sound insulation.

In the earmuff according to the present invention, the volume of the front air chamber can be made larger than that in the conventional earmuff to provide higher sound insulation. With higher degree of adhesion between the ear pad and the baffle board, the sound insulation can be further improved.

If the earmuff according to the present invention is to have the volume of front air chamber as same as that in the conventional earmuff, a baffle board to be included therein can be thinner. Thus, a thinner and lighter earmuff can be obtained.

### Second Embodiment

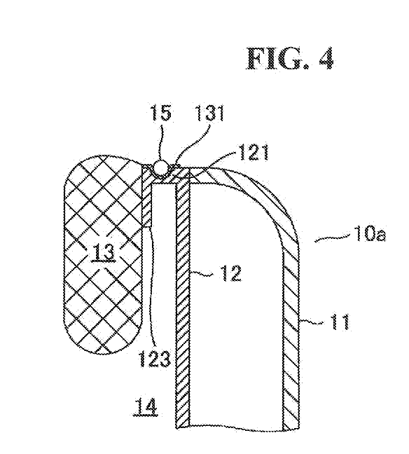

Another embodiment of an earmuff according to the present invention is described with reference to FIG. 4. An earmuff 10a illustrated in FIG. 4 includes a supporting member 123 that is an end portion of the rib 121 in the earmuff 10 described in the first embodiment, the end portion being extended in a direction towards the center of the baffle board 12.

The supporting member 123 supports the surface of the ear pad 13 facing the baffle board 12 at a certain position. Thus, a user can wear the earmuff 10a without the ear pad 13 sinking into the front air chamber 14 due to the pressure applied to the ear pad 13 in the direction towards the baffle board 12 upon wearing. In short, the supporting member 123 prevents the pressured ear pad 13 from sinking into the front air chamber 14. Therefore, the volume of the front air chamber 14 can be secured.

In the earmuff according to the present invention, the volume of the front air chamber can be made larger than that in the conventional earmuff to provide higher sound insulation. With higher degree of adhesion between the ear pad and the baffle board, the sound insulation can be further improved.

If the earmuff according to the present invention is to have a volume of front air chamber as same as that in the conventional earmuff, a baffle board to be included therein can be thinner. Thus, a thinner and lighter earmuff can be obtained.

### Third Embodiment

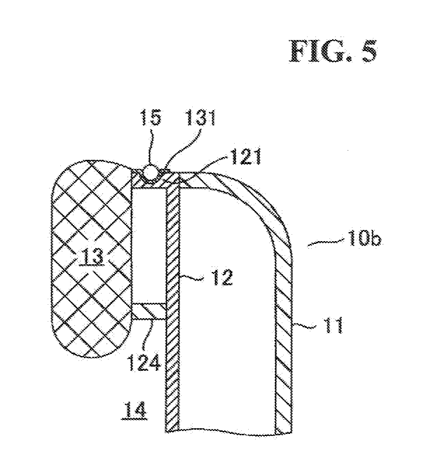

Still another embodiment of an earmuff according to the present invention is described with reference to FIG. 5. An earmuff 10b in FIG. 5 includes a supporting member 124 on the front surface of the baffle board 12 of the earmuff 10 according to the first embodiment described above.

The supporting member 124 has a shape and a size capable of supporting the surface of the ear pad 13 facing the baffle board 12. Thus, a user can wear the earmuff 10b without the ear pad 13 sinking into the front air chamber 14 due to the pressure applied to the ear pad 13 in the direction towards the baffle board 12 upon wearing. The supporting member 124 does not necessary cover the whole circumference of the baffle board 12, and should be in a size (thickness, height, and length) small enough to secure the volume of the front air chamber 14.

In the earmuff according to the present invention, the volume of the front air chamber can be made larger than that in the conventional earmuff to provide higher sound insulation. With higher degree of adhesion between the ear pad and the baffle board, the sound insulation can be further improved.

If the earmuff according to the present invention is to have a volume of front air chamber as same as that in the conventional earmuff, a baffle board to be included therein can be thinner. Thus, a thinner and lighter earmuff can be obtained.

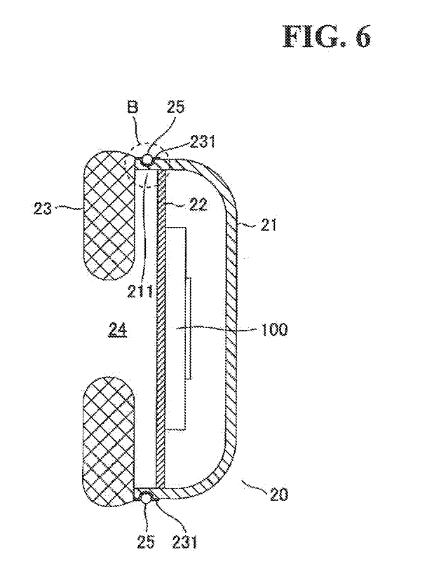

Another embodiment of an earmuff according to the present invention is described with reference to some of the figures. FIG. 6 is a longitudinal cross-sectional view of another embodiment of an earmuff according to the present invention. An earmuff 20 illustrated in FIG. 6 has its opening covered by a baffle board 22 fixed at an inner peripheral surface of an ear cup 21. A rib 211 is a portion of the outer peripheral surface of the ear cup 21 further extending outwardly from the position at which the baffle board 22 is fixed. An ear pad 23 is fixed to the rib 211. A front air chamber 24 is a space surrounded by the ear pad 23, the baffle board 22, and the rib 211.

The baffle board 22 covers the opening of the ear cup 21, and has a substantially circular shape and a size to fit the inner surface of the ear cup 21. A flap 231 is a part of a skin member of the ear pad 23 serving as an engaging unit and is fixed to the rib 211 of the ear cup 21 by being constrained by an elastic force of a ring-shaped engagement member 25.

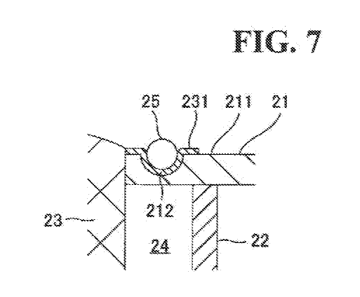

FIG. 7 is an enlarged view of an area in FIG. 6 surrounded by the dotted circle B. As illustrated in FIG. 7, a groove 212 deep enough to receive the engagement member 25 is formed on entire circumference of the rib 211. The flap 231 that is an extended portion of the skin member of the ear pad 23 covers the rib 211. The engagement member 25 is mounted on the flap 231 and is fit in the groove 212. The ear pad 23 is fixedly adhered onto the rib 211 with the engagement member 25 pressing the flap 231 against the groove 212 with its elastic force. Thus the ear pad 23 is fixed onto the ear cup 21.

As described above, the ear pad 23 can be fixed to the ear cup 21 with the volume of the front air chamber 24 not being compromised by the shape of the baffle board 22 using the engagement member 25 and the rib 211. Thus, an earmuff with an improved sound insulation can be obtained. If the volume of the front air chamber is required to be as the same as the conventional counterpart, the rib 221 can be shorter to form thinner ear cup 21. Thus, an earmuff that is thinner as a whole can be obtained.

### Fifth Embodiment

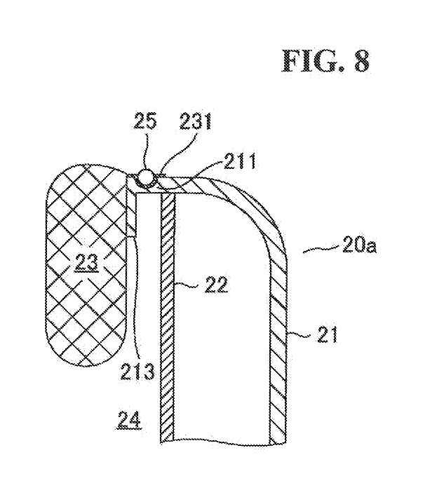

Still another embodiment of an earmuff according to the present invention is described below with reference to FIG. 8. An earmuff 20a illustrated in FIG. 8 includes a supporting member 223 that is an end portion of the rib 211 of the earmuff 20a in the forth embodiment described above, the end portion extending towards the center of the baffle board 22.

The supporting member 223 supports the surface of the ear pad 23 facing the baffle board 22 at a certain position. Thus, a user can wear the earmuff 20a without the ear pad 23 sinking into the front air chamber 24 due to the pressure applied to the ear pad 23 in the direction towards the baffle board 22 upon wearing. In short, the supporting member 223 prevents the pressured ear pad 23 from sinking into the front air chamber 24. Therefore, the volume of the front air chamber 24 can be secured.

In the earmuff according to the present invention, the volume of the front air chamber can be made larger than that in the conventional earmuff to achieve higher sound insulation. With higher degree of adhesion between the ear pad and the baffle board, the sound insulation can be further improved.

If the earmuff according to the present invention is to have a volume of front air chamber as same as that in the conventional earmuff, a baffle board to be included therein can be thinner. Thus, a thinner and lighter earmuff can be obtained.

### Sixth Embodiment

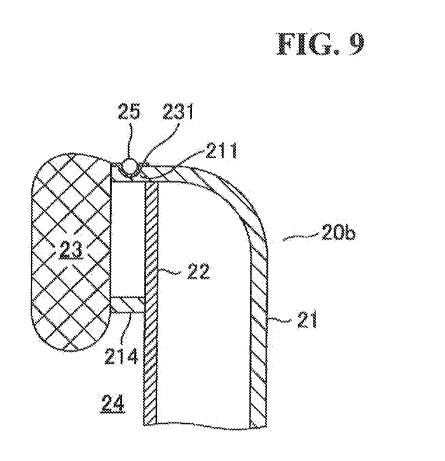

Still another embodiment of an earmuff according to the present invention is described with reference to FIG. 9. An earmuff 20b in FIG. 9 includes a supporting member 214 on the front surface of the baffle board 22 of the earmuff 20 according to the fourth embodiment described above.

The supporting member 214 has a shape and a size capable of supporting the surface of the ear pad 23 facing the baffle board 22. Thus, a user can wear the earmuff 20b without the ear pad 23 sinking into the front air chamber 24 due to the pressure applied to the ear pad 23 in the direction towards the baffle board 22 upon wearing. The supporting member 214 does not necessary cover the whole circumference of the baffle board 22, and should be in a size (thickness, height, and length) small enough to secure the volume of the front air chamber 24.

In the earmuff according to the present invention, the volume of the front air chamber can be made larger than that in the conventional earmuff to achieve higher sound insulation. With higher degree of adhesion between the ear pad and the baffle board, the sound insulation can be further improved.

If the earmuff according to the present invention is to have a volume of front air chamber same as that in the conventional earmuff, a baffle board to be included therein can be thinner. Thus, a thinner and lighter earmuff can be obtained.

### Seventh Embodiment

Yet still another embodiment of an earmuff according to the present invention is described below. Each of the flaps 131 and 231 of the earmuff according to the present invention is an outwardly extended part of the skin member of each of the ear pads 13 and 23, respectively. Instead, the flaps 131 and 231 may each be a pouch formed along an entire circumference of the ear pads 13 and 23, respectively. The engagement members 15 and 25 may be inserted in the pouch to be integrated with the flaps 131 and 231, respectively.

With the flaps 131 and 231 integrated with the engagement members 15 and 25, respectively, in the first to the sixth embodiments, flaps 131 and 231 covers and constrains the outer periphery of the ribs 121 and 211, respectively at the same time. Thus, the ear pads 13 and 23 fixedly adhere to the baffle boards 12 and the ear cup 21 without compromising the volume of the front air chambers 14 and 24, respectively.

### Eighths Embodiment

A headphone having the above sound insulation can be obtained by providing a speaker unit that outputs an audible signal on the baffle board of the earmuff according to the present invention. The embodiments illustrated in FIGS. 1 to 6 are supposed to form a headphone. The headphone can be formed by installing a speaker unit 100 on the rear surface of the baffle board 12 (22) and forming an opening through which sounds emitted by the speaker unit 100 pass on the baffle board 12 (22).

The present invention can be applied to a noise canceling headphone to provide a noise canceling headphone having an excellent sound insulation with thinner casing and lighter weight as a whole.

What is claimed is:

1. An earmuff comprising:
a casing;
a baffle board; and
an ear pad including an engagement member, the engagement member being an outwardly extended part of a skin member of the ear pad, wherein
the ear pad is fixed at a front of the baffle board with the engagement member constrained to an outer surface of either of the baffle board and the casing or both by a ring-shaped elastic member, and
the ear pad fixed and the baffle board define a front air chamber.

2. The earmuff according to claim 1, wherein a supporting member is provided at an end of the outer surface, the supporting member being in contact with a rear surface of the ear pad to prevent the ear pad form sinking into the front air chamber.

3. The earmuff according to claim 1, wherein
a supporting member having air permeability is provided on a front surface of the baffle board, and
the rear surface of the ear pad is in contact with the supporting member so that the ear pad does not sink into the front air chamber.

4. The earmuff according to claim 1, wherein a groove in which the engagement member is constrained is formed on an end of the outer surface.

5. The earmuff according to claim 1, wherein the ring-shaped elastic member and the engagement member are integrated.

6. A headphone comprising:
a speaker unit provided on a baffle board, the speaker unit outputting an audible signal, wherein
an ear pad and the baffle board is the ear pad and the baffle board in claim 1.
